(19) United States
(12) Patent Application Publication
(10) Pub. No.: US 2006/0263799 A1
(43) Pub. Date: Nov. 23, 2006

(54) Macroporous support for chemical amplification reactions

(75) Inventors: Stephan Dertinger, Munich (DE); Marco Kluehr, Munich (DE); Thomas Haneder, Dachau (DE)

Correspondence Address: DICKSTEIN SHAPIRO LLP, 1177 AVENUE OF THE AMERICAS 6TH AVENUE, NEW YORK, NY, 10036-2714, US

(73) Assignee: Infineon Technologies AG, Munich (DE)

(21) Appl. No.: 11/357,731

(22) Filed: Feb. 17, 2006

(30) Foreign Application Priority Data
Feb. 18, 2005 (EP) 05 003 588.0-2203

(51) Int. Cl. C12Q 1/68; C12P 19/34; C12M 1/34

(52) U.S. Cl. 435/6; 435/91.2; 435/287.2

(57) ABSTRACT

A method for carrying out an amplification of nucleic acids in pores of a two-dimensionally designed macroporous support material according to one embodiment includes the step of providing a predetermined part of a reaction mixture necessary for the amplification of a nucleic acid in pores of the support material. A device for carrying out the amplification of nucleic acids according to one embodiment includes a two-dimensionally designed macroporous support material having a multiplicity of pores, wherein a predetermined part of a reaction mixture for carrying out an amplification of nucleic acids is provided in the pores.

### CROSS-REFERENCE TO RELATED APPLICATION

The present application claims priority to European patent application Serial No. 05 003 588.0-2203, filed Feb. 18, 2005, which is hereby expressly incorporated by reference in its entirety.

### TECHNICAL FIELD

The present invention relates to methods for carrying out an amplification of nucleic acids in pores of a two-dimensionally designed macroporous support material, wherein a predetermined part of a reaction mixture necessary for the amplification of a nucleic acid is provided in two or more pores of the support material. The present invention furthermore relates to devices which comprise a two-dimensionally designed macroporous support material having a multiplicity of pores, wherein a predetermined part of a reaction mixture for carrying out an amplification of nucleic acids is provided in the pores.

### BACKGROUND

Methods which involve a study of nucleic acids are being used increasingly in molecular biological research, but also in the diagnosis of a wide range of diseases. To this end, the corresponding nucleic acid is generally amplified. The polymerase chain reaction (PCR) is an in vitro method for the controlled amplification of DNA fragments. Short oligonucleotides (primers) bind specifically to both ends of the intended DNA fragment and thus initiate exponential multiplication by the DNA polymerase. With 100% efficiency of the reaction, it is theoretically possible to achieve multiplication by 2ⁿ copies of the initial number of fragment copies, where n is the number of amplification cycles.

For the use of nucleic acid amplification in fundamental research, diagnosis and therapy monitoring, increasing importance is being attached to automation, parallelization and miniaturization with simultaneous improvement of the specificity. Besides the time and cost savings which are associated with automation, another great advantage is that it is possible to minimize differences in the reaction conditions for individual reaction batches due to pipetting inaccuracies or slight temperature or time variations during the amplification, or equipment-related readout inaccuracies during photometric detection of the amplification products. This means that when more components of a reaction mixture can be used for the reaction batches to be compared in a common master matrix, and when more reactions can be carried out and analyzed simultaneously, then the results will be more mutually comparable. Since PCR involves an exponential amplification of nucleic acids, minor differences in the reaction conditions are exponentiated and can therefore affect the result significantly. The specificity of established PCR conditions can be substantially increased by shortening the heating and cooling intervals of the reaction mixtures during the amplification, so as to minimize the risk of non-specific binding by the primers. Another factor in increasing the efficiency of nucleic acid amplification involves reducing the reaction volume. The smaller the reaction volume is, the faster a uniform temperature increase or decrease of the entire volume is reached. In addition, more reactions and therefore more analyses can be carried out with a sample material. Especially with limitedly available patient material, for example from a biopsy, or with sample material which requires great outlay in order to obtain it, for example cDNA, this constitutes a great advantage.

The concepts known in the prior art for miniaturizing and parallelizing the amplification of nucleic acids preferably use planar “lab-on-a-chip” arrangements (Kopp et al., Science (1998) 280, 1046-1048). These chip systems operate with a through-flow concept in which the PCR takes place continuously, with the reaction solutions travelling through different temperature zones. Besides the fact that the number of temperature cycles is established by the chip geometry, and cannot be varied, the disadvantages of this concept are a great restriction of the degree of parallelization owing to the experimental complexity of the PCR through-flow concept and the large space requirement per chip due to the planar arrangement of the PCR through-flow reactors, which can lead to high production costs. Patent Application US 2001/0055765 A1 discloses a device and a method for carrying out chemical and biochemical reactions in open reaction chambers of a planar substrate, with a multiplicity of liquid samples being placed in the individual reaction chambers, which is enormously laborious, time-consuming and cost-intensive. Like all currently existing concepts for micro- and nanotitre plates, the aforementioned US Application is based on the concept of “one reaction batch per reaction volume”. With increased miniaturization, however, the demands on dispensing technology are growing:

(i) It is necessary to dispense smaller and smaller volumes with high accuracy and high reproducibility. A further difficulty in this regard, especially in the life sciences and molecular diagnosis, is that it is often necessary to pipette reaction solutions with very different wetting properties (for example with detergents) and viscosities (for example highly concentrated protein solutions or blood), so that the accuracy and reproducibility suffer.

(ii) There are increasing requirements for the accuracy and reproducibility of the positioning of the solution to be deposited, in order to activate the individual discrete reaction vessels. Such precision dispensing systems are demanding to maintain and expensive to produce. Precision dispensing systems are unsuitable, in particular, for the service and routine laboratory sector.

Methods using a miniaturized support with corresponding reaction vessels are therefore desired, which place only minor requirements on the positioning accuracy of the dispensing device and, at the same time, allow very exact and reproducible application of very small amounts of liquid.

Accordingly, there is a need for methods and devices that offer improved conduct of the amplification of nucleic acids and overcome the above deficiencies associated with the prior art.

### SUMMARY

According to the present invention, a method is provided for amplifying nucleic acids in pores of a two-dimensionally designed macroporous support material that includes first and second surfaces lying opposite each other. A multiplicity of discrete pores with a diameter in the range of from 500 nm to 100 μm; and an aspect ratio of pore depth to pore aperture of at least 10:1; and a pore density of from 10⁴ to 10⁸/cm² are arranged and distributed over the entire surface region.

The method for amplifying the nucleic acids includes the steps of providing a predetermined part of a reaction mixture suitable for an amplification reaction in at least one region of the support material which includes at least two pores, such that part of the reaction mixture is bound non-covalently to the pore wall of the support material so that a reaction region or a reaction zone is simultaneously defined on the support material surface.

A sample is added over the entire support material, with the sample containing the part of the reaction mixture necessary for the completion of an amplification reaction. The amplification reaction is conducted and at least one amplification product is detected.

Other features and advantages of the present invention will be apparent from the following detailed description when read in conjunction with the accompanying drawings.

### BRIEF DESCRIPTION OF THE DRAWING FIGURES

The invention will be explained by way of example below with reference to the figures, in which:

FIG. 1 shows cross-sectional views (A) to (D) of various embodiments of the device according to the invention,

FIG. 2 shows a schematic plan view of an embodiment of the device according to the invention,

FIG. 3 shows cross-sectional views (A), (B) and (C) of various embodiments of conducting the amplification reaction, and

FIG. 4 shows cross-sectional views (A), (B) and (C) of various stages in the addition of a sample over all of the support material.

FIG. 5 shows cross-sectional views (A), (B), (C), (D) and (E) of various stages of one embodiment of the provision of the reaction mixture suitable for the amplification reaction on the support material.

FIG. 6 shows cross-sectional views (A), (B), (C), (D) and (E) of various stages of another preferred embodiment of the provision of the reaction mixture suitable for the amplification reaction on the support material.

### DETAILED DESCRIPTION OF THE PRESENT INVENTION

According to the present invention, a method is provided for amplifying nucleic acids in pores of a two-dimensionally designed macroporous support material that includes first and second surfaces lying opposite each other. A multiplicity of discrete pores with a diameter in the range of from 500 nm to 100 μm; and an aspect ratio of pore depth to pore aperture of at least 10:1; and a pore density of from 10⁴ to 10⁸/cm² are arranged and distributed over the entire surface region.

The method for amplifying the nucleic acids includes the steps of providing a predetermined part of a reaction mixture suitable for an amplification reaction in at least one region of the support material which includes at least two pores, such that part of the reaction mixture is bound non-covalently to the pore wall of the support material so that a reaction region or a reaction zone is simultaneously defined on the support material surface.

A sample is added over the entire support material, with the sample containing the part of the reaction mixture necessary for the completion of an amplification reaction. The amplification reaction is conducted and at least one amplification product is detected.

With an ordered porous substrate, the two requirements mentioned earlier can be satisfied when the concept of “one reaction batch per reaction volume” is discarded and a reaction batch is distributed over a plurality of mutually adjacent capillaries or pores.

A reaction region is defined not by the geometry of the support substrate but only after the application of a reaction solution or parts thereof (for example by the use of spotting techniques such as those also used for producing micro-arrays). On the porous support which is part of the present device, a reaction zone is composed of a continuous group of capillaries or pores, without adjusted spotting being necessary and the reaction zones need not lie directly next to one another, but may also be separated by intermediate spaces which comprise a plurality of pores.

A group of discrete reaction capillaries is filled owing to the suction effect of the capillary forces which occur upon contact of a solution with the porous support. Once the capillaries or pores are filled, they cannot absorb any further solutions. The porous support of the present invention is therefore a self-limiting system, with the capillary filling functioning both with blind holes and with through-holes.

There are many advantages associated with further miniaturization beyond the formats currently used. Temperature gradients over the sample can be avoided owing to the small dimensions of the support material used according to the invention. The reagent quantities which are needed in order to fill the reaction support are furthermore reduced, so that the cost per test/experiment is also reduced. Particularly in the case of chemical amplifications in which fast heating and cooling rates are required, the small dimensions of a reaction support miniaturized in this way are advantageous since the quantities of heat to be transported are small, and can therefore rapidly be pumped into the system or removed from it.

In the scope of the present invention, the term “pores” is intended to mean both pores which extend through the support material from the first surface to the second surface, i.e. through-holes, and pores which are closed at one end, i.e. blind holes. In the scope of the present invention, it is preferable to provide pores which extend through the support material from the first surface to the second surface.

The macroporous support material of the present invention is not subject to any restriction as regards material selection, so long as it comprises pores with a pore diameter of from 500 nm to 100 μm, preferably from 2 to 20 μm, and a pore density of from 10⁴ to 10⁸ pores/cm². In particular, the macroporous support material used according to the invention may be based on plastic, glass, Al₂O₃ or silicon. Preferably macroporous silicon, and even more preferably partially oxidized macroporous silicon, is used as the macroporous support material according to one embodiment of the present invention.

The macroporous silicon preferably used may be doped, preferably n-doped, or undoped. Such macroporous silicon may, for example, be produced by the method described in EP-A1-0 296 348, which is hereby incorporated by reference in its entirety. The hole openings or pores are preferably produced electrochemically, electrochemical etching being carried out in an electrolyte containing hydrofluoric acid by applying a constant or time-varying potential, the layer consisting of silicon or the substrate being connected as the positively poled electrode of an electrolysis cell. Such holes can, for example, be produced as described in V. Lehmann, J. Electrochem. Soc. 140, 1993, pages 2836 ff., which is hereby incorporated by reference in its entirety. Other semiconductor substrates, for example, such as GaAs substrates or glass substrates coated with Si₃N₄, may nevertheless also be used as a macroporous substrate of the present invention.

Partially oxidized macroporous silicon as described in WO 03/089925, to which full reference is hereby made and which is hereby incorporated by reference in its entirety, may particularly preferably be used as a macroporous support material in the scope of the present invention. Such partially oxidized macroporous silicon is intended to mean a two-dimensionally designed macroporous support material based on silicon, which includes a multiplicity of pores with a diameter in the range of from 500 nm to 100 μm, an aspect ratio of pore depth to pore aperture of at least 10:1 and a pore density of from 10⁴ to 10⁸/cm² distributed over the entire surface region. The support has at least one region which includes a plurality or multiplicity of pores with SiO₂ pore walls and this region being surrounded by a frame, arranged essentially parallel to the longitudinal axes of the pores and open towards the surface. The frame includes walls with a silicon core, with the silicon core converting into SiO₂ over the cross-section towards the outside of the walls forming the frame. Such partially oxidized macroporous silicon therefore comprises locally transparent regions of SiO₂, these transparent regions being in turn surrounded by a frame that includes walls with a silicon core. In other words, there are locally fully transparent regions of SiO₂ which are separated from one another by non-transparent walls with a silicon core, which substantially form a secondary structure.

The partially oxidized macroporous silicon used as a macroporous support material in the scope of the present invention may also be configured as described in DE 10 2004 018846.7, to which full reference is made here and which is hereby incorporated by reference in its entirety, i.e. so that each individual frame among all the frames including walls with a silicon core is spatially isolated fully from the frames surrounding it, i.e. the neighbouring frames. The silicon walls of the individual regions or compartments are then no longer continuous, i.e. they do not touch, but are fully separated from one another by regions with SiO₂ pore walls. This structure arrangement achieves spatial decoupling of the stresses locally occurring because of the volume doubling at the transition from silicon to silicon oxide in the course of the production of such partially oxidized macroporous silicon. In the fully oxidized regions, the walls between the pores are made entirely of SiO₂.

The three-dimensional arrangement of the amplification reactors, which is provided according to the invention, utilizes the depth of the substrate and allows large-scale integration of amplification reaction zones and therefore cost-effective production of the reaction support. Complete separation of the reaction volumes is achieved, so that no cross-contamination takes place. The macroporous support material according to the invention has a pore diameter of from 500 nm to 100 μm, preferably from 2 to 10 μm. This minimization of the reaction volumes to approximately 10 pl to 500 nl, preferably 1.5 to 20 nl, reduces the costs per amplification reaction. The capillary forces in the pores facilitate filling of the pores. The low heat capacity and a good thermal junction with the reaction mixture in the pore permits very fast temperature changes in the system, and therefore makes it possible to carry out selective and sensitive amplifications in a very short time with heating and cooling rates of about 3 to 10° C./s. The support material may be heated and cooled either using an external heating/cooling block (for example a heating coil/compressed air cooling or a Peltier element), preferably with a temperature sensor (for example Pt100), or using a heating element integrated on the support material (for example a heating coil), preferably with a temperature sensor (for example Pt100).

The terminology “amplification of nucleic acids” includes any enzymatic amplification of a nucleic acid, preferably by an enzyme which exhibits a polymerase activity. Examples of such enzymes are Taq polymerase or Pfu polymerase. The amplification may be carried out by a polymerase chain reaction (PCR).

Examples of a nucleic acid amplification according to the invention are real-time PCR, nested PCR, asymmetric PCR, gradient PCR, RT-PCR and methylation specific PCR.

The real-time polymerization chain reaction (real-time PCR) multiplies DNA fragments in a PCR and also allows continuous observation of the DNA amplification by measuring the fluorescence signals. This is possible both using dyes which bind nonspecifically to double-stranded nucleic acids, and by using sequence-specific probes which are bound to fluorescent dyes.

In nested PCR, a nucleic acid fragment is first amplified by means of PCR. This PCR product is used as the target nucleic acid in a second PCR. With the aid of a second primer pair, which hybridizes inside the first PCR fragment, a smaller fragment is amplified in a subsequent PCR.

The nucleic acid to be amplified may be any naturally occurring or artificially produced nucleic acid. This includes both double- and single-stranded DNA and RNA molecules and also nucleic acids with modified bases. It also includes nucleic acids which have been modified before amplification, for example by treatment with sodium bisulfite so as to convert methylated cytosine into uracil in order to detect methylated DNA in a methylation specific PCR. The amplification of DNA molecules which have previously been reverse transcribed from RNA is also included.

A predetermined part of a reaction mixture may be one or more compounds of the reaction mixture required for a nucleic acid amplification, including enzymes, nucleotides (for example deoxynucleotides such as dATP, dCTP, dGTP, dTTP, dUTP or dideoxynucleotides such as ddATP or ddCTP), salts (for example MgCl₂), primers (including primers extendable during the amplification and non-extendable primers), nucleic acids (for example DNA, RNA), PNA (peptide nucleic acid), dyes and probes.

The predetermined parts or their compounds, as explained above, of a reaction mixture suitable for the amplification reaction are usually bound onto the pore wall of the support material by drying in an aqueous solution containing these parts.

In a preferred embodiment of the present invention, the predetermined parts provided in the pores in step (a) comprise nucleotides, at least one probe and/or at least one dye, salts and primers, and also PNA in a preferred embodiment, so that the sample material to be tested and the enzyme need to be added in order to carry out the enzymatic reaction in step (c). In this way, in a particularly preferred embodiment, standardized macroscopic supports specific to a certain detection can be produced and sold to potential users. The user provides the sample in which the detection is to take place, and preferably the enzyme, and carries out the amplification of a nucleic acid.

The term “salts” covers all known salts in a concentration such that they do not prevent the enzymatic amplification of a nucleic acid.

The term “primers” covers any oligonucleotides which hybridize via base pairing with a complementary nucleic acid sequence and are used as a starting point for the addition of nucleotides. The primers of the present invention may carry a detectable marking, which makes it possible to detect the products of the nucleic acid amplification reaction.

PNAs are oligonucleotides whose bases are bound together not by a sugar-phosphate bridge but by an amino-like bridge. These constructs hybridize sequence-specifically with complementary single-stranded DNA via base pairing with a very high binding energy. Because of the bound PNA, the polymerase is not capable of amplifying the nucleic acid. As soon as mispairing takes place between PNA and nucleic acid, the binding energy decreases very greatly and the PNAs do not bind to the nucleic acid during the amplification reaction.

The term “dyes” refers to all known dyes which can be used to detect amplified nucleic acids, including fluorescent (for example intercalating) dyes such as Sybr green or ethidium bromide which bind specifically to double-stranded nucleic acids.

The term “probes” covers any known nucleotide sequence which can be used for the detection of an amplified nucleic acid by specific hybridization, including probes which are linked with a detectable marking. Such a detectable marking may, for example, be a fluorescent dye or a compound with a high affinity for a detectable marking. The probe system used in the present invention may be any known probe system which can be used for the detection of PCR products, for example TaqMan probes, molecular beacons, scorpion probes, light cycler probes or other fluorescence resonance energy transfer (FRET) probes. TaqMan probes contain a “reporter” at one end and, at the other end, a “quencher” which prevents the emission of a detectable fluorescence signal by the reporter. After hybridization of the probe with a complementary nucleic acid, the 5′→3′ exonuclease activity of the polymerase cleaves the probe so that the quencher is no longer close to the reporter, and a detectable fluorescence signal is emitted. Molecular beacons also have a “reporter” at one end and, at the other end, a “quencher” which prevents the emission of a detectable fluorescence signal by the reporter. A self-complementary sequence and the two ends of the probe leads to a hairpin structure of the probe, which is released as soon as the probe hybridizes with a complementary nucleotide sequence. The release of the hairpin structure leads to a spatial distance between the reporter and the quencher, which is sufficient for the emission of a detectable fluorescence signal by the reporter. The fluorescence resonance energy transfer (FRET) principle, on which for example light cycler probes are based, involves two probes which are complementary to neighbouring sequences of the target nucleic acid and are marked with different fluorescent dyes. After hybridization of the two probes with the target nucleic acid, the dye of one probe is stimulated and this stimulation induces the emission of a detectable fluorescence signal by the dye bound to the second probe.

The term “sample” or “sample material” covers any composition which contains an amplifiable nucleic acid. This includes artificially synthesized nucleic acids and nucleic acids isolated from viruses such as HIV, prokaryotes such as Escherichia coli, or eukaryotes such as mammals. In a preferred embodiment of the present invention, the sample material contains DNA and/or RNA which has been isolated from a human-derived biological fluid, for example blood, saliva or urine. In another preferred embodiment of the present invention, the sample material contains DNA and/or RNA which has been isolated from a human-derived tissue or tumour sample.

In one embodiment of the present invention, the sample material contains nucleic acids which have been prepared for the amplification according to the present invention. For example, preamplification of the intended nucleic acid fragment may have taken place, for example in the form of a nested PCR, or the DNA present in the sample material may have been produced by reverse transcription of an RNA, for example in the form of an RT-PCR. The nucleic acid present in the sample material may also be methylated cytosine which has been converted into uracil by a prior treatment with sodium bisulphite, for example for a methylation specific PCR. In another example, the nucleic acid present in the sample material may have been selectively enriched beforehand, for example by using probes or by chromatographic, electrophoretic or centrifugal separation.

The above methods may, for example, be used for the quantitative detection of nucleic acids. In a preferred embodiment, nucleotides, salts, at least one probe and/or at least one dye, primers and optionally PNA for the selective amplification of a particular nucleic acid fragment, are provided in step (a). Sample material, which is so dilute that one copy of the nucleic acid fragment to be amplified statistically enters a pore of the support material, may be added in step (b). If, for example, the reaction mixture contains PNAs which suppress the amplification of a particular variant of the nucleic acid fragment, for example the wild type, it is possible to detect the frequency of the modified variant, for example mutated nucleic acid. In another embodiment, the sample material may be so dilute that one genome equivalent statistically enters a pore of the support material. In this way, then it is possible to detect chromosome aberrations (for example deletions or duplications). The above examples may be employed in the diagnosis of genetic defects, new tumour diseases and in the classification of tumours. In the examples mentioned above, statistically meaningful evaluation is facilitated by the large number of reactions which can be carried out without additional working steps owing to the large number of pores of the support material being used. Depending on the mutual spacing of the pores, there may for example be from 10² to 10⁶ pores on a support material with an area of 1 mm². Another advantage of the method according to the invention is the provision of essentially equal conditions for all the reactions carried out in parallel, owing to the use of the same reaction mixture and the same sample material under equal amplification and detection conditions, so that it is possible to minimize individual variations between the individual reaction batches.

In a particularly preferred embodiment of this method, besides the primers and at least one probe and/or at least one dye for the amplification or detection of the intended nucleic acid fragment, at least one further primer pair and at least one probe specific to the nucleic acid sequence lying between these primers are also provided in step (a). Detection of the amplification product of the second primer pair with the aid of specific probes can be used as a control for the efficiency of the amplification reaction. The efficiency of the amplification reaction may also be verified by using a non-specific dye which binds the double-stranded amplification product.

In one embodiment, the method according to the invention furthermore includes providing at least one further part of a reaction mixture suitable for the amplification reaction, the composition of which differs from the composition of the part of the reaction mixture in the previous step by at least one compound or by the concentration of at least one compound, in at least one further (second) region of the support material which includes at least two pores and is different from the (first) region in the previous step. The composition of the at least one further part of a reaction mixture suitable for the amplification reaction preferably differs by at least one compound from the composition of the part of the reaction mixture in the earlier first step.

This embodiment therefore relates to a method for amplifying nucleic acids in pores of a two-dimensionally designed macroporous support material which includes a first and a second surface lying opposite each other, wherein a multiplicity of discrete pores with a diameter in the range of from 500 nm to 100 μm, an aspect ratio of pore depth to pore aperture of at least 10:1 and a pore density of from 10⁴ to 10⁸/cm² are arranged distributed over the entire surface region.

The method includes the steps of:(a) providing a predetermined part of a reaction mixture suitable for the amplification reaction in at least one first region of the support material which includes at least two pores, such that the part of the reaction mixture is bound non-covalently to the pore wall of the support material so that a reaction region or a reaction zone is simultaneously defined on the support material surface, (b) providing at least one part of a reaction mixture, the composition of which differs from the composition of the reaction mixture in step (a) by at least one compound or by the concentration of at least one compound, in at least one further second region of the support material which includes at least two pores and is different from the first region in step (a), such that the part of the reaction mixture is bound non-covalently to the pore wall of the support material, (c) adding a sample, which contains the part of the reaction mixtures of steps (a) and (b) necessary for the completion of an amplification reaction, over the entire support material, (d) conducting the amplification reaction, and (e) detecting at least one amplification product.

The individual regions may be arbitrarily defined on the porous support material.

One embodiment of the above method according to the invention is a detection method in which the presence of a particular nucleic acid in a sample material is to be checked. Since with appropriate division of the regions of the macroporous support, by providing different primers in steps (a) and (b), a multiplicity of detections can be carried out on a support material merely by varying the primers, then it is possible to test a multiplicity of known relevant biomarkers in a sample material in a single working step, for example all known tumour markers or markers for diseases which are caused by genetic defects, or a multiplicity of known gene expression markers for tumour diseases and/or metabolic diseases.

One example of such detection is the provision of nucleotides, salts, at least one probe and/or at least one dye and primers which bind specifically to the DNA of particular pathogens, for example different HIV subtypes or different HPV types, on the porous support material used according to the invention and the addition of DNA isolated from blood components and an enzyme for amplification of the DNA, for example for diagnostic purposes. Another example of such detection is the provision of nucleotides, salts, at least one probe and/or at least one dye and primers which bind specifically to the mRNA of particular genes, the expression of which is enhanced or reduced in the presence of a particular disease, on the porous support material used according to the invention, and the addition of RNA isolated from tissue or blood components and an enzyme for reverse transcription of the mRNA into DNA, and subsequent amplification of the DNA for diagnostic purposes. Another example of such detection is the detection of modified DNA, for example in order to diagnose tumour diseases, when the DNA modification detected with the aid of the method according to the invention is typical of the tumour disease. Besides nucleotides, salts, probes and/or dyes and primers in this case, PNAs which suppress the amplification of unmodified DNA may also be provided on the porous support used according to the invention. When the sample material and the appropriate enzyme are added, the user receives a positive fluorescence signal when modified DNA is present in the sample.

In another preferred embodiment of the method according to the invention, nucleotides, salts, at least one probe and/or at least one dye and various region-specific nucleic acids are provided in different regions of the support material. The remaining parts of the reaction mixture, including the same primers for all the regions, are then added, the amplification is carried out and the amplification products are detected. This embodiment may be used, for example, when the cDNAs of different tissue or tumours are provided on the support material and the intention, with the aid of the added primers, is to identify those tissues or tumours in which the nucleic acid amplified with the aid of the primers is expressed. In this way, it is possible to study the expression of known and unknown genes in various tissues, for example tissue samples from various disease stages or developmental stages, or tumours, for example different tumour types or different tumour stages.

The support material is normally sealed on one side before step (a). Before the amplification is carried out, further sealing is generally also carried out on the side which is still open, i.e. sealing is carried out on both sides. In this way, for example, it is possible to prevent evaporation of the reaction mixture when heating during the amplification reaction. The sealing may be carried out in any known way which does not substantially compromise the amplification reaction. For example, the upper or lower side of the porous support material, or both sides, may be closed with a polymer film or a mat based on elastomer (for example silicone) as a cover or seal, or the pores may be covered with a mineral oil. Another example of sealing the support material is to seal the pores with wax, microcrystalline wax, polyethylene wax or fast curing resins which do not contain any solvents or give off other chemical components that may compromise the amplification reaction.

In one embodiment of the method according to the invention, the pores may also remain unclosed on at least one side of the macroporous support material. In this case, the amplification reaction is carried out in a reaction chamber which is saturated with water vapour and whose cover can be heated.

The amplification reaction is carried out by providing conditions under which the enzyme contained in the reaction mixture amplifies a nucleic acid, preferably by cyclic heating of the support material.

The amplification product is preferably detected photometrically. The amplification product may, for example, by detected by using dyes which bind specifically to double-stranded nucleic acids or by using marked sequence-specific probes.

In one preferred embodiment of the present invention, the predetermined parts of the reaction mixture or their chemical compounds, which are introduced into the pores, are put into the pores while dissolved in a liquid (for example by using the capillary forces of the pores) and the liquid is subsequently evaporated.

In a particularly preferred embodiment of the method according to the invention, the parts which are provided from a reaction mixture suitable for the amplification reaction are applied onto the pore wall of the support material by drying-in or dehydration.

The parts which are provided from the reaction mixture suitable for the amplification reaction may be applied in the respectively intended regions of the support material by spotting methods known in the prior art.

In a preferred embodiment of the present invention, a sample is added over all of the support material in one working step. The filling of the individual pores of the support material is preferably capillary-driven. The capillary forces of the pores induce defined uniform filling of the pores, so that a self-dosing system is obtained.

A device is also provided which comprises a two-dimensionally designed macroporous support material that has a first and a second surface lying opposite each other, wherein a multiplicity of discrete pores with a diameter in the range of from 500 nm to 100 μm, an aspect ratio of pore depth to pore aperture of at least 10:1 and a pore density of from 10⁴ to 10⁸/cm² are arranged distributed over the entire surface region, wherein the device comprises at least one region in which a predetermined part of a reaction mixture for carrying out an amplification of nucleic acids is bound non-covalently in at least two pores.

A device is furthermore provided which comprises a two-dimensionally designed macroporous support material that has a first and a second surface lying opposite each other, wherein a multiplicity of discrete pores with a diameter in the range of from 500 nm to 100 μm, an aspect ratio of pore depth to pore aperture of at least 10:1 and a pore density of from 10⁴ to 10⁸/cm² are arranged distributed over the entire surface region, wherein the device includes at least one first region in which a predetermined part of a reaction mixture for carrying out an amplification of nucleic acids is bound non-covalently in at least two pores, and a second region in which a predetermined part of the reaction mixture for carrying out an amplification of nucleic acids, which differs from the first part by at least one compound, is bound non-covalently in at least two pores.

In the scope of the present invention, the term “non-covalently bound” is intended to mean the application or arrangement of chemical compounds which constitute the predetermined part of a reaction mixture suitable for the amplification reaction, as mentioned above, in or on the respective pore walls of the macroporous support material so that the compounds adhere firmly to these walls but without being linked to them by chemical covalent bonds. This firm adhesion or physisorption may be carried out by correspondingly drying-in an aqueous solution, which contains these predetermined parts or their relevant compounds. The compounds then adhere to the pore walls of the macroporous support material in a way which is reversible, indestructible and quantitatively releasable.

In the methods according to the invention, and in the devices according to the invention, a multiplicity of usually periodically arranged pores, which extend from one surface to the opposite surface of the support material, are arranged distributed over the entire surface region of the two-dimensionally designed macroporous support material. In the scope of the present invention, blind holes may be locally provided on the two-dimensionally designed macroporous support material, i.e. pores which are open on only one of the surface sides.

The macroporous support material used in the methods according to the invention and in the devices according to the invention has a pore diameter of from 500 nm to 100 μm, preferably from 2 to 20 μm. This leads to aspect ratios of pore depth to pore aperture of more than 10:1, preferably more than 40:1. The thickness of the macroporous support material is usually from 100 to 5000 μm, preferably from 300 to 600 μm. The spacing (pitch) from pore centre to pore centre, i.e. between two pores neighbouring or adjacent to each other, is usually from 1 to 100 μm, preferably from 3 to 50 μm. The pore density is usually in the range of from 10⁴ to 10⁸/cm².

The macroporous support material of the present invention is not subject to any restriction as regards material selection, so long as it includes pores with a pore diameter of from 500 nm to 100 μm, preferably from 2 to 10 μm. In particular, the macroporous support material used according to the invention may be based on plastic, glass, Al₂O₃ or silicon. Preferably macroporous silicon, and even more preferably partially oxidized macroporous silicon, is used as the macroporous support material in the scope of the present invention.

The macroporous silicon preferably used may be doped, preferably n-doped, or undoped. Such macroporous silicon may, for example, be produced by the method described in EP-A1-0 296 348. The hole openings or pores are preferably produced electrochemically, electrochemical etching being carried out in an electrolyte containing hydrofluoric acid by applying a constant or time-varying potential, the layer consisting of silicon or the substrate being connected as the positively poled electrode of an electrolysis cell. Such holes can, for example, be produced as described in V. Lehmann, J. Electrochem. Soc. 140, 1993, pages 2836 ff., which is hereby incorporated by reference in its entirety. Other semiconductor substrates, for example, such as GaAs substrates or glass substrates coated with Si₃N₄, may nevertheless also be used as a macroporous substrate in the scope of the present invention.

Partially oxidized macroporous silicon as described in WO 03/089925, to which full reference is hereby made and which is hereby incorporated by reference in its entirety, may particularly preferably be used as a macroporous support material in the scope of the present invention. Such partially oxidized macroporous silicon is intended to mean a two-dimensionally designed macroporous support material based on silicon, which includes a multiplicity of pores with a diameter in the range of from 500 nm to 100 μm distributed over the entire surface region, the support having at least one region which includes a plurality of pores with SiO₂ pore walls and this region being surrounded by a frame, arranged essentially parallel to the longitudinal axes of the pores and open towards the surface, and including walls with a silicon core, the silicon core converting into SiO₂ over the cross section towards the outside of the walls forming the frame. Such partially oxidized macroporous silicon therefore comprises locally transparent regions of SiO₂, these transparent regions being in turn surrounded by a frame including walls with a silicon core. In other words, there are locally fully transparent regions of SiO₂ which are separated from one another by non-transparent walls with a silicon core, which substantially form a secondary structure.

The partially oxidized macroporous silicon used as a macroporous support material in the scope of the present invention may also be configured as described in DE 10 2004 018846.7, to which full reference is made here and which is hereby incorporated by reference in its entirety, i.e. so that each individual frame among all the frames including walls with a silicon core is spatially isolated fully from the frames surrounding it, i.e. the neighbouring frames. The silicon walls of the individual regions or compartments are then no longer continuous, i.e. they do not touch, but are fully separated from one another by regions with SiO₂ pore walls. This structure arrangement achieves spatial decoupling of the stresses locally occurring because of the volume doubling at the transition from silicon to silicon oxide in the course of the production of such partially oxidized macroporous silicon. In the fully oxidized regions, the walls between the pores are made entirely of SiO₂.

The pores in the methods according to the invention, and in the devices according to the invention, may for example be configured essentially round or elliptically. If in particular a partially oxidized macroporous silicon is used as the macroporous support material in the scope of the present invention, then the pores with SiO₂ pore walls may be designed so that they are essentially square. The frames include walls with a silicon core may then be essentially square or rectangular in shape.

The use of a two-dimensionally designed macroporous support material as described above for the amplification of nucleic acids is furthermore provided.

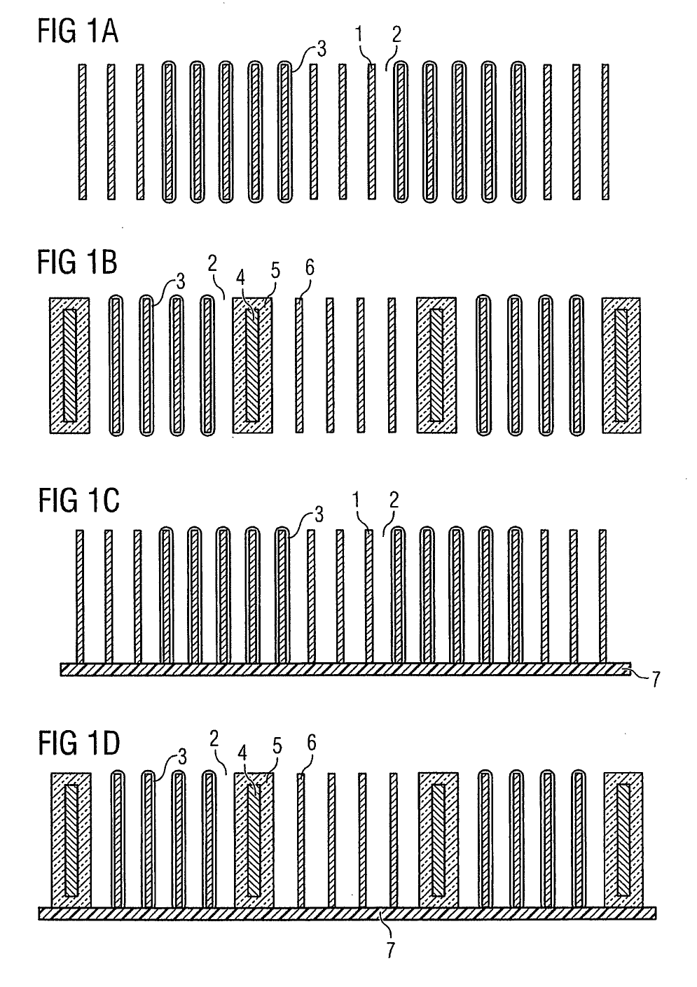

The various embodiments of the present invention will be understood with reference to the present drawing figures. FIG. 1 shows a cross-sectional view (A) of an embodiment of a device according to the invention based on a macroporous support material, for example glass, Al₂O₃ or silicon, wherein (1) represents a pore wall, (2) represents a pore and (3) represents dried-in compounds forming the predetermined part of a reaction mixture suitable for the amplification reaction; (B) a cross-sectional view of an embodiment of a device according to the invention based on partially oxidized macroporous silicon, wherein (3) represents dried-in compounds forming the predetermined part of a reaction mixture suitable for the amplification reaction, (4) represents the silicon core of a pore wall, (5) represents SiO₂ and (6) represents an SiO₂ pore wall; a cross-sectional view of the embodiment shown in (A) with a covering (7) based for example on elastomer arranged on the lower surface side, which functions as a seal, and (D) a cross-sectional view of the embodiment shown in (B) with a covering (7) arranged on the lower surface side.

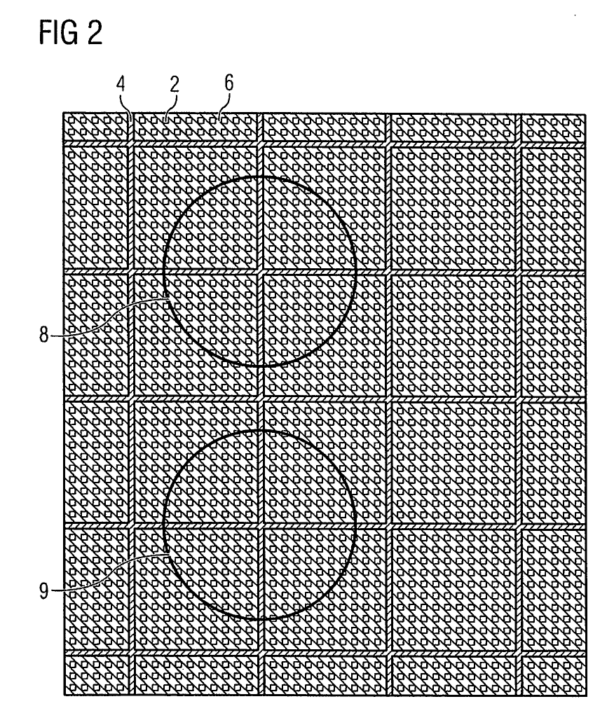

FIG. 2 shows a schematic plan view of an embodiment of a device according to the invention based on partially oxidized macroporous silicon, wherein (2) represents a pore, (4) represents a pore wall with a silicon core, (6) represents an SiO₂ pore wall (8) represents a first region of the support material in which a predetermined part of a reaction mixture suitable for the amplification reaction is provided and (9) represents a second region of the support material in which a predetermined part of a reaction mixture, which differs by at least one compound from the part of the reaction mixture suitable for the amplification reaction provided in the first region, is provided.

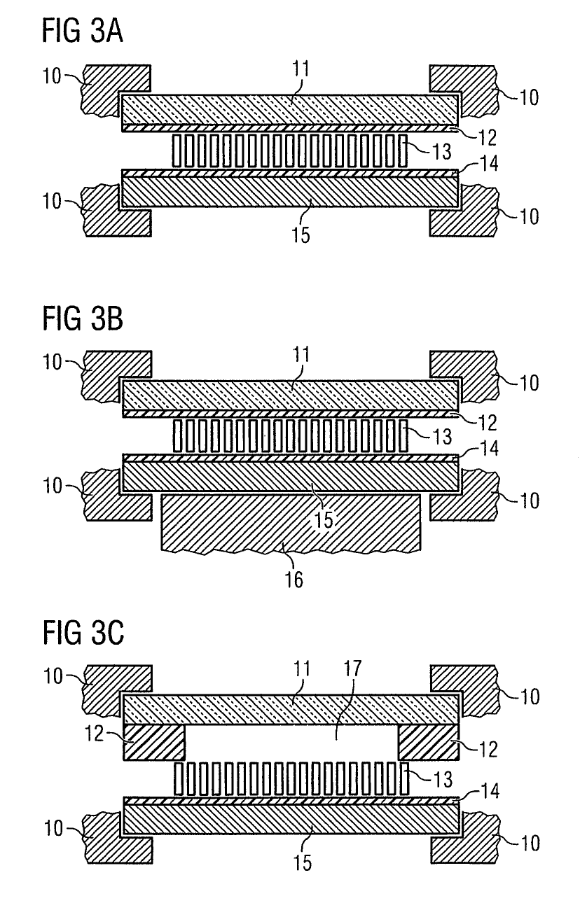

FIG. 3 shows a cross-sectional view (A) of an embodiment of a way of carrying out the amplification reaction, wherein (10) represents a holder (for example of plastic) with a low heat capacity, (11) represents a transparent window (for example of glass), (12) represents a chemically inert seal of from 1 to 200 μm (for example a silicone elastomer, for example of polydimethylsiloxane), (13) represents a device according to the invention, (14) represents a chemically inert seal of from 1 to 200 μm (for example a silicone elastomer, for example of polydimethylsiloxane), (15) represents a reflective, non-light scattering, chemically inert base with a high thermal conductivity (for example silicon), the holder pressing together the window and the base in order to achieve a seal; (B) a cross-sectional view of the embodiment shown in (A) with a heating and/or cooling block (16) and (C) a cross-sectional view of the embodiment shown in (A) with a reaction chamber (17) over the device according to the invention.

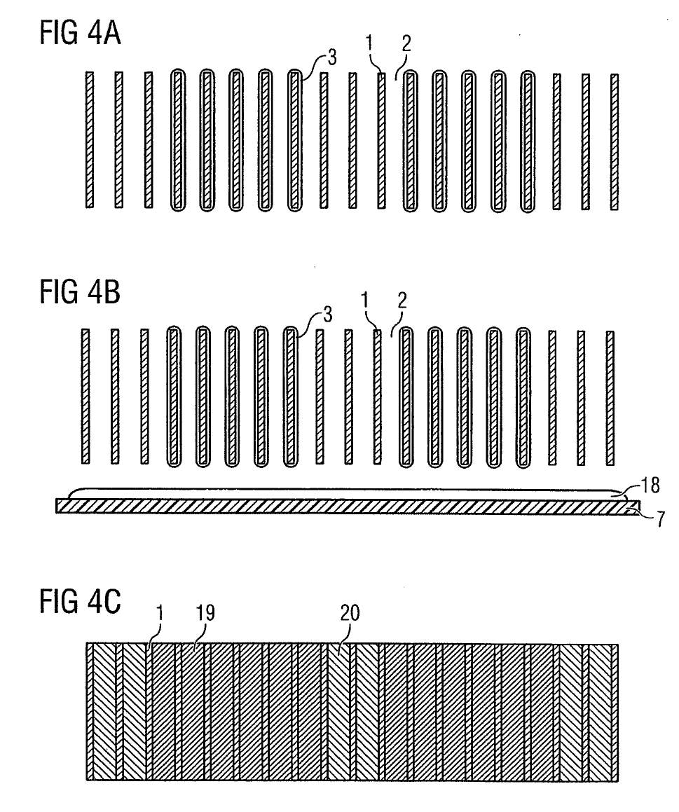

FIG. 4 shows a cross-sectional view of various stages in the addition of a sample over all of the support material, (A) showing a cross-sectional view of an embodiment of a device according to the invention, wherein (1) represents a pore wall, (2) represents a pore and (3) represents dried-in compounds forming the predetermined part of a reaction mixture suitable for the amplification reaction; (B) showing the cross-sectional view of an embodiment of a device according to the invention and a covering (7) with a sample material (18) applied on the surface side and (C) showing the cross-sectional view of an embodiment of a device according to the invention after addition of the sample, (19) being a pore which is filled with a reaction mixture suitable for the amplification and which lies in a reaction region defined on the support material surface and (20) being a pore which contains only the part of the reaction mixture necessary for the completion of an amplification reaction, so that no amplification takes place in such a pore, and which lies between the reaction regions defined on the support material surface.

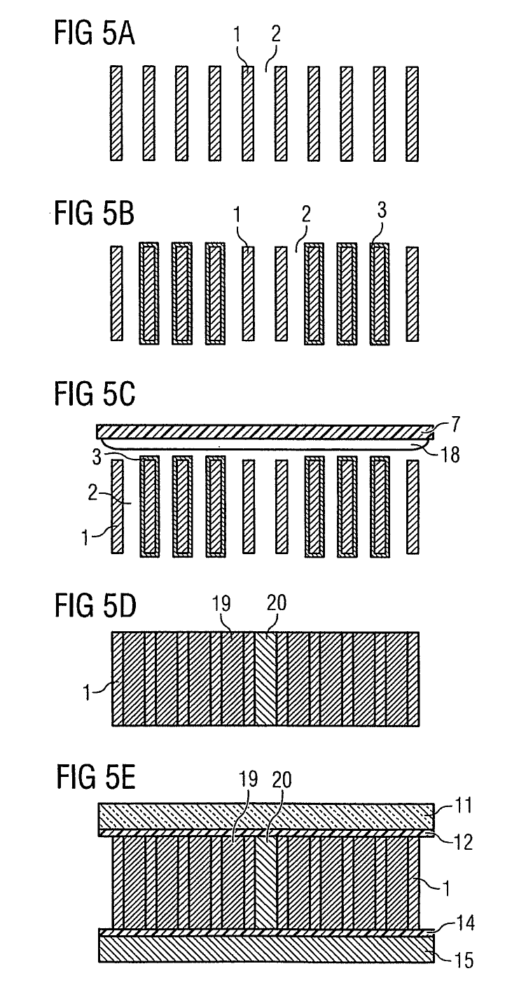

FIG. 5 shows a cross-sectional view of an embodiment of the provision of the reaction mixture suitable for the amplification reaction on the support material, (A) a cross-sectional view of an embodiment of a device according to the invention, wherein (1) represents a pore wall, (2) represents a pore; (B) the cross-sectional view of an embodiment of a device according to the invention after the provision of a predetermined part of a reaction mixture suitable for the amplification reaction in at least one region of the support material, such that the part of the reaction mixture is bound non-covalently to the pore wall of the support material so that a reaction region is thereby simultaneously defined on the support material surface, (3) representing dried-in compounds; (C) the cross-sectional view of an embodiment of a device according to the invention and a covering (7) with a sample material (18) applied on the surface side and (D) the cross-sectional view of an embodiment of a device according to the invention after addition of the sample, (19) being a pore which is filled with a reaction mixture suitable for the amplification and which lies in a reaction region defined on the support material surface and (20) being a pore which contains only the part of the reaction mixture necessary for the completion of an amplification reaction, so that no amplification takes place in such a pore, and which lies between the reaction regions defined on the support material surface; (E) the cross-sectional view of an embodiment of a way of carrying out the amplification reaction, wherein (11) represents a transparent window (for example of glass), (12) represents a chemically inert seal of from 1 to 200 μm (for example a silicone elastomer, for example of polydimethylsiloxane), (14) represents a chemically inert seal of from 1 to 200 μm (for example a silicone elastomer, for example of polydimethylsiloxane) and (15) represents a reflective, non-light scattering, chemically inert base with a high thermal conductivity (for example silicon).

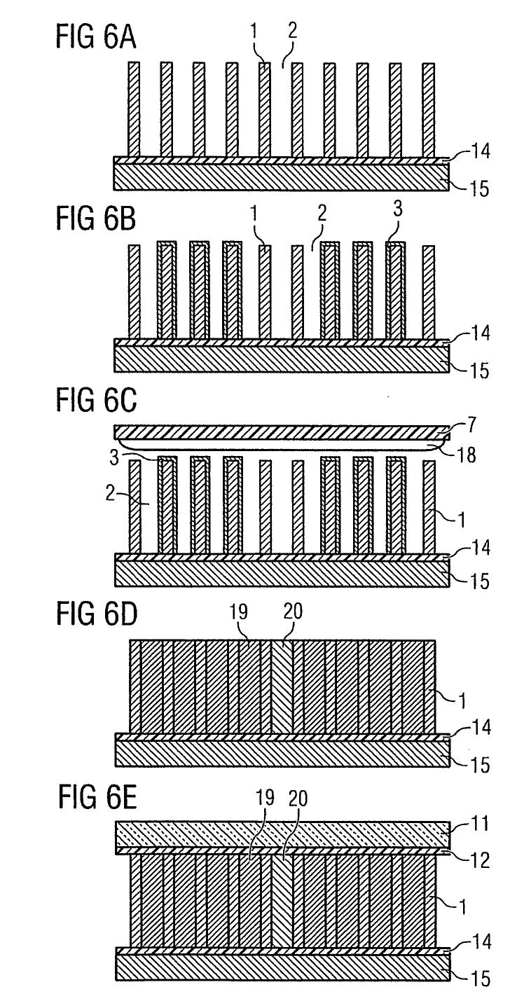

FIG. 6 shows a cross-sectional view of another preferred embodiment of the provision of the reaction mixture suitable for the amplification reaction on the support material, (A) showing a cross-sectional view of a preferred embodiment of the device according to the invention, wherein (1) represents a pore wall, (2) represents a pore, (14) represents a chemically inert seal of from 1 to 200 μm (for example a silicone elastomer, for example of polydimethylsiloxane) and (15) represents a reflective, non-light scattering, chemically inert base with a high thermal conductivity (for example silicon); (B) showing the cross-sectional view of an embodiment of a device according to the invention after the provision of a predetermined part of a reaction mixture suitable for the amplification reaction in at least one region of the support material, such that the part of the reaction mixture is bound non-covalently to the pore wall of the support material so that a reaction region is thereby simultaneously defined on the support material surface, (3) representing dried-in compounds; (C) showing the cross-sectional view of an embodiment of a device according to the invention and a covering (7) with a sample material (18) applied on the surface side and (D) showing the cross-sectional view of an embodiment of a device according to the invention after addition of the sample, (19) being a pore which is filled with a reaction mixture suitable for the amplification and which lies in a reaction region defined on the support material surface and (20) being a pore which contains only the part of the reaction mixture necessary for the completion of an amplification reaction, so that no amplification takes place in such a pore, and which lies between the reaction regions defined on the support material surface; (E) showing the cross-sectional view of an embodiment of a way of carrying out the amplification reaction, wherein (11) represents a transparent window (for example of glass) and (12) represents a chemically inert seal of from 1 to 200 μm (for example a silicone elastomer, for example of polydimethylsiloxane).

While exemplary drawings and specific embodiments of the present invention have been described and illustrated, it is to be understood that the scope of the present invention is not to be limited to the particular embodiments discussed. Thus, the embodiments shall be regarded as illustrative rather than restrictive, and it should be understood that variations may be made in those embodiments by workers skilled in the art without departing from the scope of the present invention as set forth in the claims that follow, and equivalents thereof. In addition, the features of the different claims set forth below may be combined in various ways in further accordance with the present invention.

1. A method for amplifying nucleic acids in pores of a two-dimensionally designed macroporous support material which includes first and second surfaces lying opposite each other, wherein a multiplicity of discrete pores with a diameter in the range of from 500 nm to 100 μm, an aspect ratio of pore depth to pore aperture of at least 10:1, and a pore density of from 10⁴ to 10⁸/cm² are arranged distributed over an entire surface region, wherein the method comprises the steps of:
(a) providing a predetermined part of a reaction mixture suitable for the amplification reaction in at least one region of the support material which includes at least two pores, such that the part of the reaction mixture is bound non-covalently to the pore wall of the support material so that a reaction region is thereby simultaneously defined on the support material surface;
(b) adding a sample over the entire support material, the sample containing the part of the reaction mixture necessary for the completion of an amplification reaction;
(c) conducting the amplification reaction; and
(d) detecting at least one amplification product.

2. The method according to claim 1, further comprising the step of: providing at least one further part of a reaction mixture suitable for the amplification reaction, the composition of which differs from the composition of the part of the reaction mixture in step (a) by at least one compound or by the concentration of at least one compound, in at least one second region of the support material which comprises at least two pores and is different from the region in step (a).

3. The method according to claim 1, wherein the predetermined parts of a reaction mixture suitable for the amplification reaction have been bound to the pore wall of the support material by drying-in an aqueous solution containing these parts.

4. The method according to claim 1, wherein the macroporous support material is one of a macroporous silicon and partially oxidized macroporous silicon.

5. A device comprising a two-dimensionally designed macroporous support material which includes first and second surfaces lying opposite each other, wherein a multiplicity of discrete pores with a diameter in the range of from 500 nm to 100 μm, an aspect ratio of pore depth to pore aperture of at least 10:1, and a pore density of from 10⁴ to 10⁸/cm² are arranged and distributed over an entire surface region, wherein the device comprises at least one region in which a predetermined part of a reaction mixture for carrying out an amplification of nucleic acids is bound non-covalently in at least two pores.

6. The device according to claim 5, wherein the device comprises at least one further region in which a predetermined part of the reaction mixture for carrying out an amplification of nucleic acids, the composition of which differs from the composition of the part of the reaction mixture in the first region by at least one compound, is bound non-covalently in at least two pores.

7. The device according to claim 5, wherein one surface side of the support material is closed by a covering.

8. The device according to claim 5, wherein the macroporous support material is one of macroporous silicon and partially oxidized macroporous silicon.

9. The device according to claim 5, wherein the pores have a diameter in the range from 2 to 20 μm.

10. The device according to claim 5, wherein the support material has a thickness of between 100 and 5000 μm.

11. A method for amplification of nucleic acids that includes incorporating into a device a two-dimensionally designed macroporous support material which comprises first and second surfaces lying opposite each other, wherein a multiplicity of discrete pores with a diameter in the range of from 500 nm to 100 μm, an aspect ratio of pore depth to pore aperture of at least 10:1, and a pore density of from 10⁴ to 10⁸/cm² are arranged and distributed over an entire surface region.

12. The method according to claim 11, further including the steps of:
disposing the device between a first holder and a second holder such that the device is securely held in place therebetween, with the device being inertly sealed along first and second faces thereof that face the first and second holders, respectively;
providing a transparent window between the first face of the device and the first holder; and
providing a reflective, non-light scattering base between the second face of the device and the second holder.

13. The method according to claim 12, further including the step of:
pressing the first and second holders such that the window and the base are pressed together resulting in the device being sealed.

14. The method according to claim 13, further including the step of:
providing a reaction chamber above the first face of the device and between the window and the device.
